(19) United States
(12) Patent Application Publication
(10) Pub. No.: US 2011/0212411 A1
(43) Pub. Date: Sep. 1, 2011

(54) SYSTEM AND METHOD FOR OPTICAL FIBER DIFFUSION

(76) Inventor: Edward L. Sinofsky, Dennis, MA (US)

(21) Appl. No.: 13/126,962

(22) PCT Filed: Oct. 30, 2009
(86) PCT No.: PCT/US09/62803

§ 371 Date: Apr. 29, 2011

Related U.S. Application Data
(60) Provisional application No. 61/197,863, filed on Oct. 31, 2008.
(60) Provisional application No. 61/197,860, filed on Oct. 31, 2008.
(60) Provisional application No. 61/110,309, filed on Oct. 31, 2008.

(51) Int. Cl. A61N 5/06; G02B 6/26; A61B 1/24; G02B 1/12

(52) U.S. Cl. 433/29; 385/27; 607/88; 264/1.27

(57) ABSTRACT

An optical fiber diffusion system and a method of manufacturing an optical fiber diffusion device that has a precisely-controlled emission region are disclosed. An optical fiber diffusion device is produced by subjecting a light emission region of an optical fiber to a series of controlled cycles of stress, heating, elongation and cooling, resulting in a pattern of deformation and modification of the fiber and cladding.

### RELATED APPLICATIONS

This application claims the benefit of U.S. Provisional Application No. 61/197,860, filed on Oct. 31, 2008, and claims the benefit of U.S. Provisional Application No. 61/197,863, filed on Oct. 31, 2008, and claims the benefit of U.S. Provisional Application No. 61/110,309, filed on Oct. 31, 2008. The entire teachings of the above applications are incorporated herein by reference.

### BACKGROUND OF THE INVENTION

Fiber optic diffusion systems have been used in a wide number of applications including, but not limited to, architectural and decorative lighting, photographic and microscopic illumination, the polymerization of industrial polymers, and endoscopic, dental and catheter-based instruments used to deliver optical radiation to a targeted biological site from within a body lumen or cavity.

Conventional diffusing tips typically consist of a standard fiber optic strand terminating in a diffusing region that incorporates an overtube, which increases the diffuser diameter to the outer dimension of the overtube. Such a conventional construction has several drawbacks. First, using an overtube of a larger diameter than the optical fiber increases the minimum lumen diameter through which the optical fiber device can pass. Next, from an optical point of view, there are the reflection and absorption losses in the transmission power, which may be transferred to the overtube. Mechanically, the overtube causes an abrupt change in stiffness that can cause kinking when the optical fiber device is bending through complex curves. Overtubes also must be adhered well to the fiber to avoid detaching during use. Further, the overtube adds additional component costs, manufacturing steps and related expenses.

In conventional diffusers, a means for extracting the light out of the fiber core is typically formed either by abrading or removing the fiber's cladding, or by injecting the light out of the distal end of the fiber into a polymer mixture of a Many of these conventional diffusing tip designs rely on a reflective end mirror to define the distal end of the diffuser, as well as acting to homogenize the intensity distribution along the tip. This end mirror has been typically placed in the distal end of the overtube. Although this approach works well, it has many detrimental properties and limitations. First and foremost of such drawbacks is the high cost and skill involved in fabricating end mirrors, especially small ones. The end mirrors must be precisely ground and polished to optical standard to be able to accept the optical coating, either metallic or dielectric, which is typically deposited on the end face. If this mirror is used to homogenize the light output by obtaining a second optical pass through the diffusing media, or a second optical pass down the cladding stripped fiber strand, some of the retro-reflected light transmits back down the fiber and is lost, which lowers the optical efficiency of the diffusing tip.

Certain conventional techniques involve the removal of all or part of the fiber's cladding by solvent, acid or abrasion of the cladding. These are complicated procedures. To produce a uniform light distribution by cladding removal, one either needs to use a gradient of abrasion or etch the cladding to a uniform thickness on the order of an optical wavelength. Either approach requires very high precision, and special facilities designed with complex equipment and safety procedures. Glass fibers typically become weakened when subjected to cladding manipulation and or removal, which could cause catastrophic failure in the field.

Other conventional techniques rely on the injection of light from the distal face of an optical fiber into a matrix polymer that contains a carefully controlled amount of scattering sites. One either needs to make the scattering sites have a gradient along the tip, or interact with an end mirror to make a substantially uniform light distribution. These manufacturing techniques are also complicated and costly, and may have low manufacturing yields due to bubble formation in the matrix during the assembly and curing of the tip. Degassing the matrix before injection into the tip helps increase yield, but adds significant time and cost to the manufacturing process.

The diffusion tips made with such a conventional technique also have the problem of optical and mechanical damage at the fiber/epoxy interface. This interface is subject to burning-like failures as well as to mechanically induced shearing damage when the fiber is bent at that interface.

In addition, the cost of conventional diffusing tips has hindered their widespread use in medicine.

### SUMMARY OF THE INVENTION

As the above described optical fiber optical diffusive devices have proven less than optimal, it is an object of an embodiment according to the present invention to provide an improved diffusive optical device with a precise, stable, controlled illumination over a predefined region.

It is a further object of an embodiment according to the invention to provide an improved optical diffusive device that is highly efficient.

A further object of an embodiment according to the present invention is to provide optical diffusive devices that may be constructed from a single continuous fiber without the need for an overtube.

It is a further object of an embodiment according to the invention to provide an improved optical diffusive device with a fiber optic emission region having a diameter equal to or less than the transmitting fiber.

A further object of an embodiment according to the present invention is to provide an optical diffusive device that inhibits the effects of heat cycling.

A further object of an embodiment according to the present invention is to provide optical diffusive devices that are simple and inexpensive to manufacture without the need for an end mirror.

A further object of an embodiment according to the present invention is to provide optical diffusive devices that have a non-binding, flexible tip.

Another object of an embodiment according to the present invention is to provide a disposable diffusing tip that is coupled to a reusable dental handpiece containing a reusable fiber optic cable.

A further object of an embodiment according to the present invention is to provide near infrared light transmission to a therapeutic site with a combination of glass fiber optic cable and a polymer diffusing tip, such as by delivering the light to a handpiece with glass fiber and then diffusing with a polymer diffusing tip.

A further object of an embodiment according to the present invention is to provide substantially uniform illumination at the surface of a balloon catheter, even though the optical diffuser has a non-uniform illumination pattern on its surface.

An embodiment according to the present invention provides a method of manufacturing an optical fiber diffusion device that has a precisely-controlled emission region. This is accomplished by subjecting the designated emission region to a series of controlled cycles of stress, heating, elongation and cooling, which results in a pattern of deformation and modification of the fiber and cladding.

The manufacturing process may be precisely controlled, in accordance with an embodiment of the invention, by precisely monitoring the amount of optical radiation exiting the optical fiber at each emission region during manufacture of the optical fiber, using a sensor affixed to the distal terminus of the fiber. Such a method in accordance with an embodiment of the invention may be beneficially applied to the construction of a multiplicity of closely-spaced emission sub-regions with defined emission patterns, thus enabling precisely uniform illumination of designated objects.

The resulting optical fiber diffusion device in accordance with an embodiment of the invention achieves substantially improved levels of uniformity, flexibility and durability, while remaining within the dimensional envelope of the original optical fiber.

Another advantage of an embodiment according to the present invention over conventional devices is that the device may be constructed from a single fiber, which obviates the alignment and integrity problems of conventional devices; and enables a stable, uniform beam in a durable construction unaffected by the extreme thermal cycling of sterilization and other treatments.

An embodiment according to the present invention provides a method of manufacture in which the precise emission of the optical fiber, or of a sub-region of the optical fiber, may be dynamically established by monitoring the output of the distal fiber terminus. The change in the distal terminus transmission inversely correlates to the light emission in the effected sub-region of active manufacture.

A further embodiment according to the invention provides the ability to manufacture a series of distinct sub-regions of light emission, which may be designed to emit a uniform illumination at a given radial distance, or at the surface of the diffuser. One application of such an embodiment is the illumination of a balloon catheter. The radiation extracted from the distinct bands of light emission may integrate to produce a uniform illumination at the surface of the balloon.

Another embodiment according to the invention provides a low cost, disposable diffusing tip that can be easily coupled to a reusable fiberoptic handpiece; such as by coupling a disposable fiber diffuser to a reusable dental handpiece.

In accordance with one embodiment of the invention, there is provided an optical fiber diffusion device. The device comprises an optical fiber including a proximal terminus arranged to be coupled to a radiant energy source, and a distal terminus region including at least one light emission region arranged to emit light from the optical fiber. The at least one light emission region includes at least one crazed diffusion feature formed in the material of the optical fiber itself.

In further, related embodiments, the at least one light emission region may comprise a plurality of discrete light emission sub-region bands, each light emission sub-region band of the plurality including at least one crazed diffusion feature formed in the material of the optical fiber itself. The at least one light emission region may comprise a plurality of discrete optical sub-regions arranged to emit a substantially equal amount of light from each discrete optical sub-region of the plurality. The optical fiber may comprise a polymer material. The at least one light emission region may comprise optical fiber cladding that is not abraded, and may comprise optical fiber cladding none of which is chemically removed. The at least one light emission region may have the same or a smaller diameter than the diameter of the optical fiber. The at least one light emission region may comprise at least one elongated emission region. The optical fiber diffusion device may comprise no mirror, and may comprise no overtube. Further, the at least one light emission region may comprise a plurality of heat-effected light emission sub-regions, each light emission sub-region including necking and crazing of the optical fiber. In addition, the at least one light emission region may comprise a plurality of light emission sub-regions having logarithmic sub-region spacing. The at least one crazed diffusion feature may be the result of heating and elongating the fiber. The at least one crazed diffusion feature may be of a configuration that emits light in a fashion that provides substantially uniform illumination of at least one designated object. The at least one light emission region may comprise a plurality of discrete light emission sub-region bands, each light emission sub-region band of the plurality including at least one crazed diffusion feature formed in the material of the optical fiber itself, the plurality of discrete light emission sub-region bands being arranged in said configuration that emits light in a fashion that provides substantially uniform illumination of at least one designated object.

In other related embodiments, the optical fiber diffusion device may further comprise a catheter coupled to the optical fiber, the catheter including a balloon illuminated by light from the optical fiber. The at least one light emission region may comprise a plurality of discrete light emission sub-region bands being separated from each other by a distance approximately equal to or less than a radius of the balloon.

In another embodiment according to the invention, there is provided a method for the manufacture of an optical diffusion device. The method comprises the steps of: (a) applying a stress to a portion of an optical fiber that includes a location of a light emission region to be formed in the optical fiber; (b) applying thermal radiation to a sub-region of the portion of the optical fiber that includes the location of the light emission region to be formed in the optical fiber, until a deformation of the sub-region occurs; and (c), repeating steps (a) and (b) for at least one additional sub-region of the portion of the optical fiber to produce the light emission region in the optical fiber, the light emission region comprising a plurality of discrete light emission sub-region bands formed by the applying of the stress and the applying of the thermal radiation.

In further, related embodiments, the method may further comprise, prior to the applying the stress and the applying thermal radiation: affixing a radiant source to a proximal terminus of the optical fiber; affixing an optical transmission sensor to a distal terminus of the optical fiber; clamping the optical fiber at a first proximal position between the radiant source and the location of the light emission region to be formed in the optical fiber; and clamping the optical fiber at a first distal position between the optical transmission sensor and the location of the emission region to be formed in the optical fiber. The method may also comprise controlling at least one of the applying the stress and the applying thermal radiation based on an amount of light transmitted from a distal end of the optical fiber. The controlling may be performed based on monitoring the amount of light transmitted from the distal end of the optical fiber to achieve a desired light emission from an effected sub-region of active manufacture, said controlling being based on inversely correlating the amount of light transmitted from the distal end of the optical fiber versus the desired light emission from the effected sub-region of active manufacture. A thermal emitter may be moved along the optical fiber to apply the thermal radiation to the at least one additional sub-region. The thermal radiation may be applied using a thermal emitter from the group consisting of: a heat gun, a radio frequency device, a light device, a soldering tip, a laser, a coil, and an ultrasound device.

In another embodiment according to the invention, there is provided a method for the manufacture of an optical diffusion device from a continuous roll of optical fiber. The method comprises rolling the optical fiber out of a source roll around which the optical fiber is rolled, such that a region of the optical fiber that is to be formed into at least one light emission region is positioned within a plurality of manufacturing devices to be used in manufacturing the optical diffusion device; and monitoring light emitted from the fiber during manufacturing using a sensor coupled to the optical fiber. The sensor may be coupled to a distal terminus of the optical fiber. The sensor may be rotatable, and the method may further comprise receiving the manufactured optical diffusion device using a continuous uptake roll around which manufactured optical fiber is rolled. The monitoring may comprise monitoring the amount of light transmitted from the distal end of the optical fiber to achieve a desired light emission from an effected sub-region of active manufacture, said monitoring being based on inversely correlating the amount of light transmitted from the distal end of the optical fiber versus the desired light emission from the effected sub-region of active manufacture. The method may comprise manufacturing an optical fiber diffusion device comprising the optical fiber, the optical fiber diffusion device comprising: the optical fiber, the optical fiber including a proximal terminus arranged to be coupled to a radiant energy source, and a distal terminus region including the at least one light emission region, the at least one light emission region being arranged to emit light from the optical fiber and comprising a plurality of discrete light emission sub-region bands, each light emission sub-region band of the plurality including at least one crazed diffusion feature formed in the material of the optical fiber itself.

In another embodiment according to the invention, there is provided an optical fiber diffusion device. The device comprises an optical fiber including a proximal terminus arranged to be coupled to a radiant energy source, and a distal terminus region including at least one light emission region arranged to emit light from the optical fiber, the at least one light emission region including at least one crazed diffusion feature formed in the material of the optical fiber itself; and a catheter coupled to the optical fiber, the catheter including a balloon illuminated by light from the optical fiber.

In further, related embodiments, the at least one light emission region may comprise a plurality of discrete light emission sub-region bands being separated from each other by a distance approximately equal to or less than a radius of the balloon. The at least one crazed diffusion feature may be of a configuration that emits light in a fashion that provides substantially uniform illumination of the balloon. The at least one light emission region may comprise a plurality of discrete light emission sub-region bands, each light emission sub-region band of the plurality including at least one crazed diffusion feature formed in the material of the optical fiber itself, the plurality of discrete light emission sub-region bands being arranged in said configuration that emits light in a fashion that provides substantially uniform illumination of the balloon.

In another embodiment according to the invention, there is provided a method of treating the human body. The method comprises introducing an optical fiber diffusion device into a vascular vessel of the human body, the optical fiber diffusion device comprising an optical fiber including a proximal terminus arranged to be coupled to a radiant energy source, and a distal terminus region including at least one light emission region arranged to emit light from the optical fiber, the at least one light emission region including at least one crazed diffusion feature formed in the material of the optical fiber itself; and illuminating the optical fiber diffusion device.

In further, related embodiments, the optical fiber diffusion device may further comprises a catheter coupled to the optical fiber, the catheter including a balloon illuminated by light from the optical fiber. The method comprises performing a balloon angioplasty.

In another embodiment according to the invention, there is provided an optical fiber diffusion device. The device comprises a source optical fiber including (i) a proximal terminus of the source optical fiber arranged to be coupled to a radiant energy source, and (ii) a distal terminus of the source optical fiber; and an emission optical fiber including a proximal terminus of the emission optical fiber coupled to the distal terminus of the source optical fiber, the emission optical fiber comprising a distal terminus region including at least one light emission region arranged to emit light from the emission optical fiber, the at least one light emission region including at least one crazed diffusion feature formed in the material of the emission optical fiber itself.

In further, related embodiments, the emission optical fiber may comprise a disposable tip. The source optical fiber may be reusable. The device may comprise a handpiece of a dental tool, the handpiece including at least a portion of the source optical fiber. The emission optical fiber may be detachably coupled to the source optical fiber. The at least one light emission region may comprise a tapered tip. The emission optical fiber may comprise a polymer. The source optical fiber may comprise a glass fiber.

In another embodiment according to the invention, there is provided a method of providing treatment light from an optical therapeutic system. The method comprises diffusing light from an optical fiber diffusion device in or near a target treatment region of a patient, the optical fiber diffusion device comprising a source optical fiber including (i) a proximal terminus of the source optical fiber arranged to be coupled to a radiant energy source, and (ii) a distal terminus of the source optical fiber; and an emission optical fiber including a proximal terminus of the emission optical fiber coupled to the distal terminus of the source optical fiber, the emission optical fiber comprising a distal terminus region including at least one light emission region arranged to emit light from the emission optical fiber, the at least one light emission region including at least one crazed diffusion feature formed in the material of the emission optical fiber itself.

In further, related embodiments, the method may comprise detachably coupling the emission optical fiber to at least one therapeutic light output fiber of the optical therapeutic system, the at least one therapeutic light output fiber comprising the source optical fiber. The optical fiber diffusion device may be incorporated in an applicator of the optical therapeutic system. The emission optical fiber may be used external to the body of the patient, and/or incorporated into a surgical instrument for internal use, and/or introduced into a body cavity of the patient.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a diagram of an optical fiber diffusion system according to an embodiment of the invention.

FIG. 2 is a side view of a light emission region of an optical fiber diffusion system in accordance with an embodiment of the invention.

FIG. 3A is a side view of a distal terminus of a light emission region of an optical fiber diffusion system in accordance with an embodiment of the invention.

FIG. 3B is a diagram of an optical fiber with a tapered distal terminus, in accordance with an embodiment of the invention.

FIG. 4 is a graph of a relationship between light emission and light transmission in an optical fiber diffusion system according to an embodiment of the invention.

FIGS. 5A-5E are diagrams of steps in a process for manufacturing an optical fiber diffusion system, in accordance with an embodiment of the invention.

FIGS. 6A and 6B are diagrams of a method of continuous, automated manufacture of an optical fiber diffusion system, in accordance with an embodiment of the invention.

FIG. 7 is a side view of an optical fiber diffusion system used with a balloon catheter, in accordance with an embodiment of the invention.

FIG. 8A is a graph of emission power in the emission region of an optical fiber diffusion system in accordance with an embodiment of the invention.

FIG. 8B is a graph of light intensity at the surface of a balloon catheter, in accordance with an embodiment of the invention.

FIGS. 9A-9B are diagrams of an optical fiber diffusion system using a disposable optical fiber light emission region, in accordance with an embodiment of the invention.

### DETAILED DESCRIPTION OF THE INVENTION

A description of example embodiments of the invention follows.

An embodiment according to the invention provides an optical diffusion system, and in particular provides a fiber optic diffusion device having precisely controlled light emission from intermediate regions or termini of the fiber.

In accordance with an embodiment of the invention, a monolithic diffusing tip that has no overtube enables the full use of the available fiber transmission diameter, improves the durability of the instrument, and with a slight tapering of the tip provides improved bending and tracking characteristics.

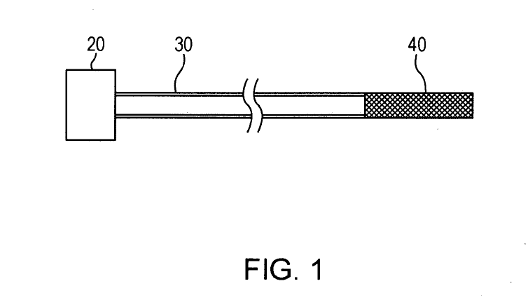

FIG. 1 is a diagram of an optical fiber diffusion system according to an embodiment of the invention. The system includes a light or electromagnetic radiation source 20, an optical fiber 30 and a light or radiation emission region 40. For photo-optical applications, the light source 20 is often a fiber-coupled laser source and may span the spectrum from UV to infrared. Single or multiple wavelengths of light may be simultaneously employed.

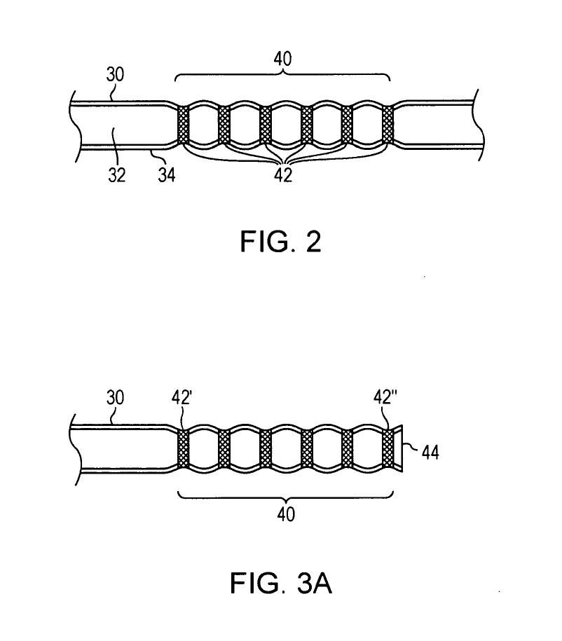

FIG. 2 is a side view of a light emission region 40 in an optical fiber diffusion system in accordance with an embodiment of the invention. As will be described further below, the optical fiber 30, having a core 32 and cladding 34, is transformed into a series of sub-regions 42 having a distorted scattering architecture which permits the emission of a precise amount of light at each sub-region 42. The individual sub-regions 42 may be separated by unmodified optical fiber or may be constructed as a continuous series. In a given series, each sub-region 42 may be constructed with unique characteristics, including but not limited to axial emission length, emissivity per unit area, emissivity per steradian, spatial emissivity distribution, and deformation profile (cylindrical axial “necking”). The dark sub-regions 42 shown in FIG. 2 may, for example, be regions of striated or crazed features formed in the material by the application of heat and stress to the material, as discussed further below. The regions of crazing may appear white when the material is cooled, depending on the material used for the fiber. The crazing features scatter light to produce diffusion when light is transmitted through the optical fiber 30. The crazing features are formed in the material of the optical fiber 30 itself and are directly bounded by the surrounding space into which the optical fiber diffusion device is to emit light, rather than being surrounded by or bounded by any overtube, end mirror, scattering matrix or any other interface. By avoiding the need the use such added interfaces to diffuse light, an optical fiber diffusion device according to an embodiment of the invention avoids a number of drawbacks of conventional optical fiber devices.

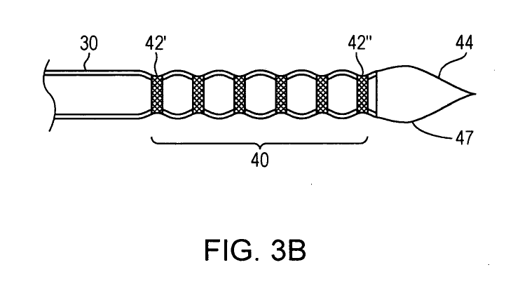

FIG. 3A is a side view of an optical fiber 30 that is cut at the distal terminus 44 of the emission region 40, in accordance with an embodiment of the invention. The distal terminus 44 may be flat, tapered or shaped as appropriate. The optical fiber 30 includes emission sub-regions extending from proximal sub-region 42′ to distal sub-region 42″. It will be understood that the light emission sub-regions 42 may be positioned in any pattern or position along the optical fiber 30, and may be constructed abutting each other to provide a continuous emission region 40. Such an arrangement may be optimal in some applications, while the arrangement of sub-regions 42 of the embodiment of FIG. 3A may be useful in others.

FIG. 3B is a diagram of an optical fiber 30 with a tapered distal terminus 44, in accordance with an embodiment of the invention. As with the embodiment of FIG. 3A, the optical fiber 30 includes an emission region 40 with emission sub-regions extending from proximal sub-region 42′ to distal sub-region 42″. A radiused end 47 with tapered tip 44 combines to improve tracking, for example when the optical fiber 30 is used in a catheter, by preventing the device from hanging up as it moves through the catheter.

In an embodiment according to the invention, in the case where the double integral of irradiance from the emission sub-regions of the optical fiber on the surface of an enclosing cylinder is a constant, the average axial emission at each equally spaced sub-region 42 must also be an equal value. However, since the optical radiation is principally injected at the proximal terminus, the emissivity as a percentage of the fiber transmission beam at the first or proximal sub-region 42′ (see the embodiment of FIG. 3A) must be less than that of the distal sub-region 42″. The relevant formula is the fractional series 1/10, 1/9, ⅛, . . . 1/1 for a ten sub-region emission fiber, whereby the proximal sub-region 42′ emits 1/10th of the 10 unit beam or one unit of the beam power, the next sub-region 1/9th of the remaining 9 unit beam or one unit of the beam power, and so forth until the distal sub-region 42″ emits 1/1 of the remaining 1 unit beam or the last remaining one unit of the beam power.

Among the many advantages of an embodiment according to the invention is the providing of precise light emission from a continuous fiber and the elimination the losses at coupling interfaces. Another advantage of an embodiment according to the invention is that the diameter of the fiber at the light emission region 40 is the same or smaller than the diameter of the rest of the fiber. This feature facilitates the precise placement of the fiber, and, for example, reduces the impact of insertion and removal on tissues when the optical fiber is used in operating on the human body. This feature also produces a fiber diffuser that because of its mechanical design is both trackable and pushable.

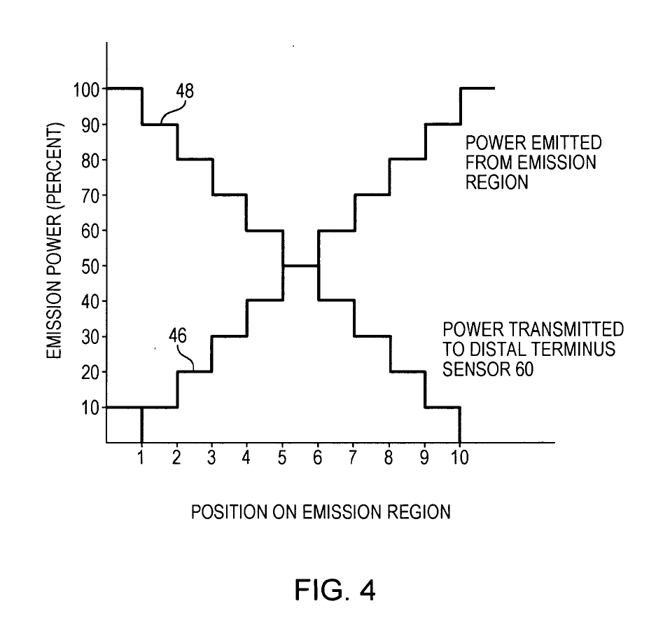

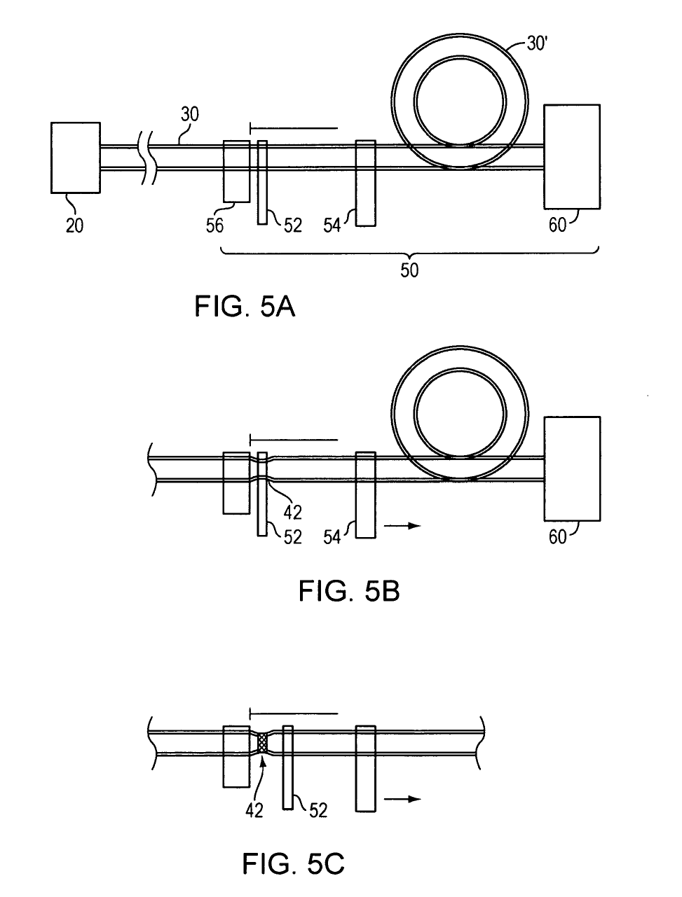

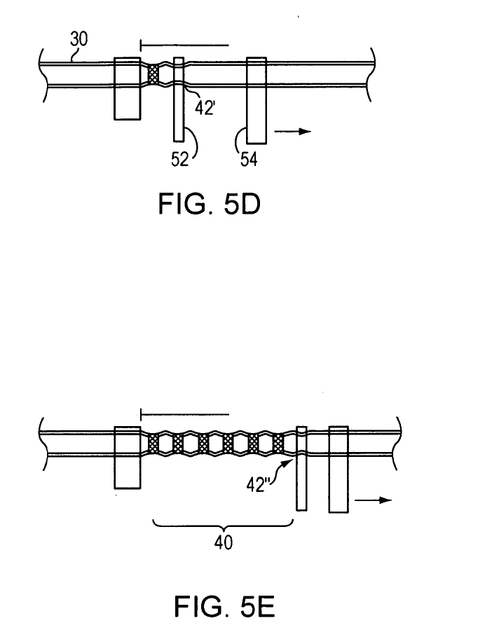

FIG. 4 is a graph of a relationship between light emission 46 and light transmission 48 in an optical fiber diffusion system according to an embodiment of the invention. The graph shows the light emission/transmission percentage versus sub-region steps over a ten band emission region with a uniform emission profile. The abscissa (x-axis) of the graph of FIG. 4 represents the position of the sub-region steps along the emission region 40 (see the embodiment of FIG. 3A), from the position of the proximal sub-region 42′ (on the left of the x-axis of FIG. 4) to the position of the distal sub-region 42″ (on the right of the x-axis of FIG. 4). The ordinate (y-axis) of the graph of FIG. 4 represents light intensity (emission power). Two quantities are graphed: quantity 46 is the power emitted from each emission sub-region 42 (see FIG. 3A), while quantity 48 is the power transmitted to a transmission intensity sensor 60 (see FIG. 5A, described below) positioned at the distal terminus of the optical fiber. At the zero point on the abscissa, corresponding to the virtual interface between the transmitting optical fiber 30 and the emission region 40 (see FIG. 3A), quantity 48 shows that one hundred percent of the normalized optical fiber transmitted light would be recorded at the intensity sensor 60. For an unmodified optical fiber in which no sub-regions 42 have yet been formed, the properties of total internal reflection continue to transmit this normalized level of one hundred percent to the distal optical transmission sensor 60. Upon the construction of the first light emission sub-region 42, the amount of light which is extracted in this sub-region 42 is subtracted from the amount transmitted to the distal sensor 60. There is a nearly linear correlation between the amount of light extracted from the sub-regions 42 (shown as quantity 46 in FIG. 4) and the amount subtracted from the light transmitted to the distal sensor 60 (shown as quantity 48 in FIG. 4). This correlation may be used as a precise feedback loop during manufacturing of the optical fiber, as described below in connection with FIGS. 5A-5E.

The graph of the embodiment of FIG. 4 shows that as the emission sub-regions 42 are added towards the distal end of emission region 40, the amount of light extracted increases (see quantity 46), while the amount of light transmitted to the sensor 60 decreases proportionally (see quantity 48). In this representation, equal amounts of light are extracted in steps at each of ten discrete sub-regions 42, but any pattern may be manufactured including but not limited to continuous, parametric, discrete and combinations thereof.

FIGS. 5A-5E are diagrams of steps in a process for manufacturing an optical fiber diffusion system, in accordance with an embodiment of the invention.

In the embodiment of FIG. 5A, an optical fiber 30 having a radiation source 20 mounted to its proximal end is positioned across a manufacturing apparatus 50, with the distal end of the optical fiber positioned at an optical fiber transmission intensity sensor 60. The transmission level of light to the sensor 60 may be monitored throughout the manufacturing process, and an initial reference level is recorded. It will be understood that a portion of the fiber 30 may form one or more loops 30′. The optical fiber 30 is held stationary by a proximal clamp 56 and placed under stress by actuated clamp 54, the force on which is indicated by an arrow. The thermal unit 52 applies heat to the optical fiber 30 while the actuated clamp 54 continues to apply stress. When the stress deformation temperature of the optical fiber 30 is reached in the sub-region that is being formed, as a result of heating by thermal unit 52, the optical fiber 30 will deform and elongate, resulting in a “necking” of the fiber and a transformation in the geometry, structure and continuity of the fiber core 32 and cladding 34 interface (shown in FIG. 2). The result is an increase in the emission of radiation from the deformed or emission sub-region 42 (see FIG. 5B) and simultaneously an equal decrease in the radiation monitored by the sensor 60. By controlling the prescribed level of stress on the fiber 30 through the actuated clamp 54, the emission from the sub-region 42 may be precisely established. When the design level of emission from the sub-region 42 is reached, the heat is removed and the thermal unit 54 is re-positioned at the next sub-region.

FIG. 5B shows the “necking” of the first sub-region 42 following the application of heat from the thermal unit 52 and stress from the actuated distal clamp 54, in accordance with an embodiment of the invention. In accordance with the discussion of the graph of FIG. 4, in an embodiment of the invention, the amount of light transmitted to the distal sensor 60 may be used to closely monitor the dynamic “necking” and transformation of the sub-region 42 from a total internal reflective state to a controlled emission sub-region 42. The sub-regions 42 may be formed to have a light emission profile that is consistent with the emission graph of FIG. 4. Other emission profiles may be generated.

FIG. 5C shows the movement of the thermal unit 52 to the next sub-region, in accordance with an embodiment of the invention. In the manufacturing process of an embodiment according to the invention, a multiplicity of factors may be controlled and monitored to facilitate the optimal method to be used for a given application of the optical fiber system. For example speed and simplicity may be balanced against the precision and quality of the manufacturing. The movement and thermal profile of the thermal unit 52, as well as the parameters of a cooling element that may be used, provides three additional degrees of freedom in this process. Other control factors will be apparent to those of skill in the art.

FIG. 5D shows the re-application of stress to the fiber 30 by the actuator clamp 54 with the thermal unit 52 having been moved to the next sub-region 42′, in accordance with an embodiment of the invention.

FIG. 5E shows the completed emission region 40 prior to cutting, in accordance with an embodiment of the invention. Sub-region 42″ is the most distal of the emission sub-regions.

In accordance with an embodiment of the invention, the light transmitted to the sensor 60 may be monitored by a human monitoring the transmission level measured by the sensor 60, or by automated control devices. The stress applied to the optical fiber by the distal clamp 54 may be controlled by a human monitoring the stress applied; and/or by using a spring-loaded micrometer of other measurement tool; and/or by using automated control devices. The heat applied by the thermal unit 52 may similarly be controlled by human monitoring, and/or by thermal instrumentation, and/or by using automated control devices. Generally, the heat applied by the thermal unit should be sufficient to produce a desirable degree of crazing or similar phenomenon in the optical fiber material, which may occur slightly below the melting point of the fiber material. If the fiber is heated too much, smooth melting may occur, which may not produce sufficient crazing of the material and may produce insufficient scattering of light off the resulting regions formed in the fiber. On the other hand, it is necessary to heat the fiber enough that crazing can occur. The amount of time that stress is applied to the fiber may also be controlled: the longer that stress is applied to the fiber by the distal clamp 54, the deeper the crazing features that are formed. Therefore, the amount of time may be varied to produce crazing features of the desired depth. Other control techniques may be used. In accordance with embodiments of the invention, automated control devices for implementing techniques described herein may include, for example, mechanical, electrical, optical and thermal sensors and devices, and associated electronics, instrumentation, and data processing hardware. It will be appreciated that human monitoring may replace or supplement automated control devices for implementing such techniques.

In accordance with an embodiment of the invention, an optical fiber may be formed, for example, from a polymer, such as from a plastic material, such as an acrylic poly (methyl methacrylate) (PMMA) fiber. Such materials have the advantage of low price compared to glass fibers. An optical fiber fabricated using techniques according to an embodiment of the invention has the advantage of reducing expense by comparison with optical fiber diffusion devices that use end mirrors as part of the diffusion tip. Plastic materials have the further advantage of not cracking, and remaining flexible in use in a variety of applications where flexibility is desirable.

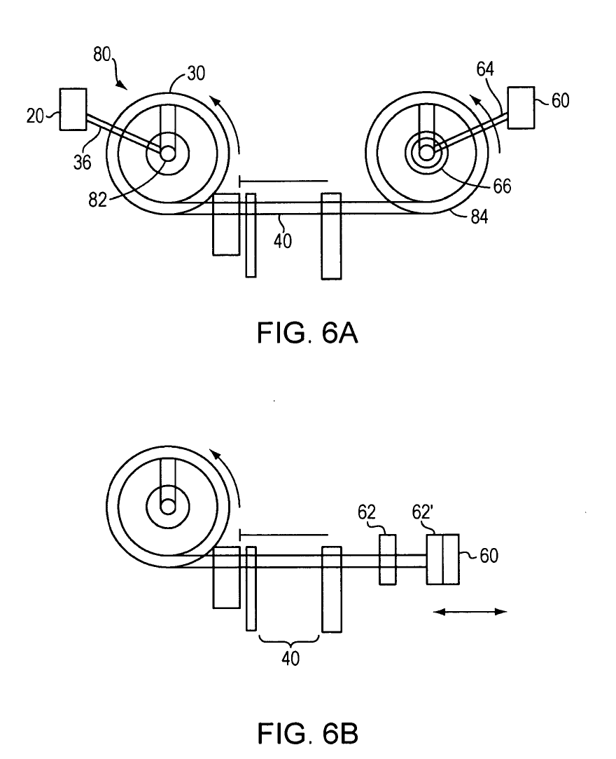

FIGS. 6A and 6B are diagrams of a method of continuous, automated manufacture in accordance with an embodiment of the invention.

In the embodiment of FIG. 6A, a continuous roll manufacturing method permits a long continuous roll of optical fiber 30 to be continuously fed into the elements that are used to manufacture the emission region 40. A radiant source 20 is coupled to a proximal portion 36 of the optical fiber 30. The proximal portion 36 of the fiber leads into a center coupling 82, around which the remainder of the fiber 30 forms a feed roll 80. The continuous roll system may use a rotating optical coupling 82, a data/sensor power slip ring assembly or a wireless transmitter to couple the fiber to the radiant source 20. The sensor 60 may be coupled to an uptake roll 84 of the optical fiber in a similar manner to the way in which the radiant source 20 is coupled to the feed roll 80. For example, a distal end 64 of the fiber may lead out from a central coupling 66 (such as a slip ring) of the uptake roll 84 to the sensor 60. This embodiment may be advantageously employed for the manufacture of continuous rolls of fiber having spaced emission regions for many applications including but not limited to continuous rolls to be cut into discrete elements; therapeutic wraps, bandages and garments; architectural, safety and ornamental lighting; industrial radiant sources for sensors and measuring devices; and other applications. In particular, the embodiment of FIG. 6A may be used when a single long optical fiber is used, having long spacings between separate emission regions along the fiber.

In the embodiment of FIG. 6B, a continuous source roll is used to produce discrete elements, in a similar fashion to that described for FIG. 6A. One or more fiber cutters 62 may be employed. In operation a portion of fiber having a completed emission region 40 is drawn by movable sensor 60 and first cut by proximal cutter 62, and if desired by an additional distal cutter 62′. The sensor 60 then returns to its operational position to be coupled with the next portion of the fiber to be cut. In a similar fashion to the embodiment of FIG. 6A, the radiant source may be coupled to the optical fiber using a rotating coupling; or the source electronics may be connected to the rolled fiber using a slip ring assembly. The embodiment of FIG. 6B may be used, for example, to mass produce separate optical fiber devices in each of which a single emission region 40 (see FIG. 3A) features multiple closely-spaced emission sub-regions 42.

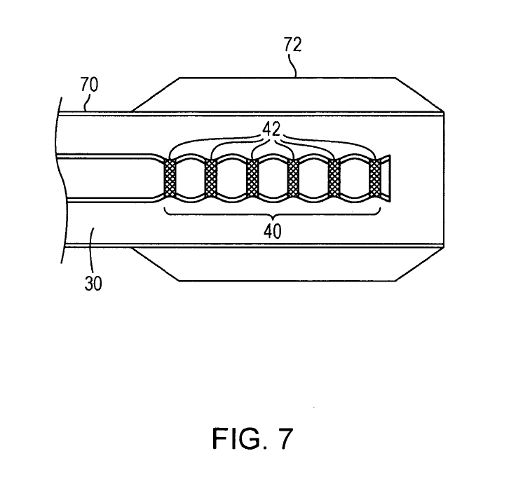

FIG. 7 is a side view of an optical fiber diffusion system used with a balloon catheter, in accordance with an embodiment of the invention. A balloon 72 of the catheter assembly 70 encloses the emission region 40 of the optical fiber 30. In one embodiment, the spacing between the individual emission sub-regions 42 is approximately equal to the radial distance from the surface of each sub-region 42 to the surface of the balloon 72. This helps to ensure a uniform illumination of the surface of the balloon 72. Such a balloon catheter device may be used, for example, to perform a balloon angioplasty operation, or for example in any other setting in which it is desirable to displace liquid or tissue with an inflated balloon that emits light. Such a device may be used, for example, in a variety of different possible cavities or lumens of the human body, such as in the prostate, in tumors, in the repair of a blood vessel, in the fallopian tubes, or in other cavities or lumens. The balloon 72 may be formed, for example, from a translucent or transparent material, such as polyethylene terephthalate (PETE), urethane or other materials.

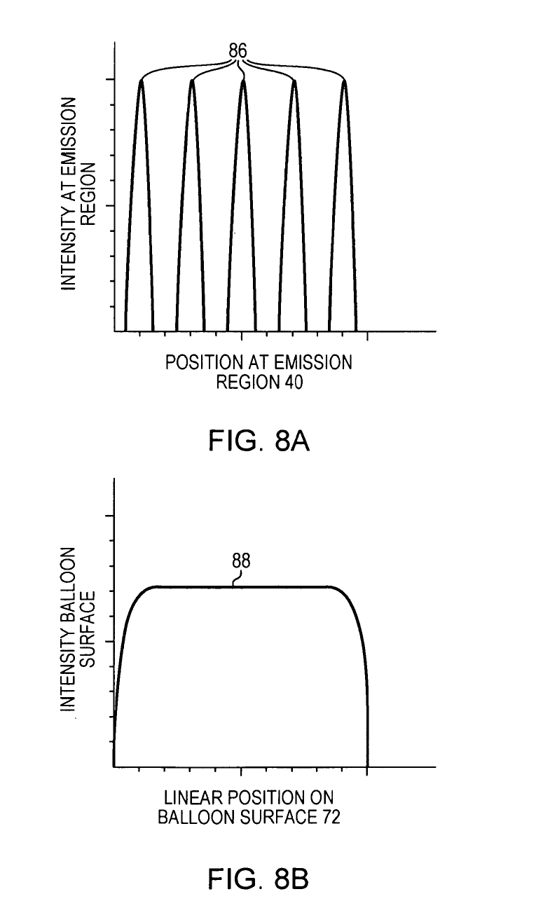

FIG. 8A is a graph of the emission power in the emission region 40 (see FIG. 7) of an optical fiber diffusion system in accordance with an embodiment of the invention. The y-axis gives the emission power at the emission region, and the x-axis gives the position along the emission region. As can be seen, emission peaks 86 are present when the emitted light is measured at the surface of the emission region.

FIG. 8B is a graph of light intensity at the surface of a balloon catheter, in accordance with an embodiment of the invention. The y-axis gives the light intensity as measured at the balloon surface, and the x-axis gives the linear position on the balloon surface 72. The light from the emitting sub-regions 42 (see FIG. 7) is integrated at the surface of the balloon 72 (FIG. 7) to produce a smoothly uniform intensity 88 (FIG. 8B) when the light is measured at the balloon surface. This may be of advantage, for example, in providing a uniform illumination of a cavity or lumen when the balloon catheter is used in the human body. In one embodiment, the emission sub-regions of the optical fiber may be manufactured such that the light emission peaks 86 of FIG. 8A have an approximately equal height, and therefore integrate to foiin a uniform intensity 88 when the light is measured at the balloon surface as shown in FIG. 8B. Further, if the spacing between the individual emission sub-regions 42 (FIG. 7) is approximately equal to the radial distance from the surface of each sub-region 42 to the surface of the balloon 72, it will help to ensure a uniform illumination of the surface of the balloon 72. Wider spacings between the emission sub-regions may prevent a uniform illumination 88 of the surface of the balloon. A spacing, for example, of 1.5 mm may be used, although it will be apparent that other spacings may be used.

In another embodiment according to the invention, a logarithmic spacing between emission sub-regions may be used. For example, at one end of the emission region 40 (see FIG. 3A), the most distal or proximal of the sub-regions 42 may be spaced apart by a distance A, where A is the base number of the logarithmic spacing; after which subsequent spacings between sub-regions 42, as one moves away from such distal or proximal end, may be equal to A^{N} with N progressing in a series such as 2, 3, 4, . . . etc. until the final spacing between sub-regions is reached. Other spacing arrangements may be used.

In another embodiment according to the invention, an optical fiber diffusion system according to an embodiment of the invention may be used for photoactivation of compounds and biomaterials. Other embodiments may generally be used in a variety of different possible cavities or lumens of the human body, such as in the prostate, in tumors, in the repair of a blood vessel, in other vascular applications, in the biliary duct, in the urinary tract, in the urethra, in the bladder, in the bladder neck, in the fallopian tubes, in the nasal cavity or in other cavities or lumens.

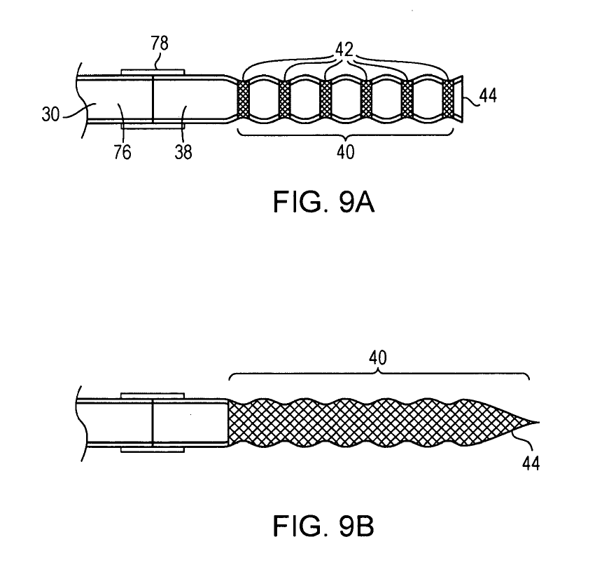

FIGS. 9A-9B are diagrams of an optical fiber diffusion system using a disposable optical fiber light emission region 40, in accordance with an embodiment of the invention. The distal terminus 44 of the emission region 40 may be tapered, as shown in FIG. 9B. The light emission region 40 may be constructed from a polymer material and may be flexible. The light emission region 40 may be made from, for example, a standard acrylic PMMA fiber, a fluoropolymer-based fiberoptic, or a polymer tube made with fluoropolymers to enhance near infrared transmission, fabricated in a similar fashion to those described elsewhere herein, including emission sub-regions 42. The disposable emission region 40 is coupled to the source fiber 30 through coupling 78 wherein the exit terminus of the source fiber 76 and the entry terminus 38 of the emission region 40 are aligned. If region 40 is the same diameter or larger than the fiber 30 diameter this coupling 78 can be made with little loss.

In an embodiment according to the invention, a low cost disposable diffusing tip is coupled to a reusable dental handpiece containing a reusable fiber optic cable. For example, such a coupling may be made using the embodiment of FIGS. 9A-9B. Near infrared light transmission (or light transmission in another region of the spectrum) may be provided to a therapeutic site with a combination of glass fiber optic cable and a polymer diffusing tip, such as by delivering the light to a handpiece with glass fiber and then diffusing with a polymer diffusing tip. For example, source fiber 30 of the embodiment of FIG. 9A may be the glass fiber optic cable through which the light is delivered to the handpiece, while disposable emission region 40 of FIG. 9A is the polymer diffusing tip. Such an embodiment may be used in dental and other therapeutic applications.

In various embodiments, diffusion tips of the type described herein may be used to diffuse treatment light from optical therapeutic systems, for example, the therapeutic systems described in the following United States patents and patent application Publications: U.S. Pat. No. 7,470,124 (“Instrument for delivery of optical energy to the dental root canal system for hidden bacterial and live biofilm thermolysis”); U.S. Pat. No. 7,255,560 (“Laser augmented periodontal scaling instruments”); U.S. Pat. App. Pub. No. 20090118721 (“Near Infrared Microbial Elimination Laser System (NIMELS)”); U.S. Pat. App. Pub. No. 20090105790 (“Near Infrared Microbial Elimination Laser Systems (NIMELS)”); U.S. Pat. App. Pub. No, 20090087816 (“Optical Therapeutic Treatment Device”); U.S. Pat. App. Pub. No. 20080267814 (“Near Infrared Microbial Elimination Laser Systems (NIMELS) for Use with Medical Devices”); U.S. Pat. App. Pub. No. 20080159345 (“Near Infrared Microbial Elimination Laser System”); U.S. Pat. App. Pub. No. 20080139992 (“Near-infrared electromagnetic modification of cellular steady-state membrane potentials”); U.S. Pat. App. Pub. No. 20080138772 (“Instrument for Delivery of Optical Energy to the Dental Root Canal System for Hidden Bacterial and Live Biofilm Thermolysis”); U.S. Pat. App. Pub. No. 20080131968 (“Near-infrared electromagnetic modification of cellular steady-state membrane potentials”); U.S. Pat. App. Pub. No. 20080077204 (“Optical biofilm therapeutic treatment”); U.S. Pat. App. Pub. No. 20080058908 (“Use of secondary optical emission as a novel biofilm targeting technology”); U.S. Pat. App. Pub. No. 20080021370 (“Near Infrared Microbial Elimination Laser System”); U.S. Pat. App. Pub. No. 20080008980 (“Laser augmented periodontal scaling instruments”); U.S. Pat. App. Pub. No. 20040156743 9 (“Near infrared microbial elimination laser system”); and U.S. Pat. App. Pub. No. 20040126272 (“Near infrared microbial elimination laser system”).

For example, in some embodiments, the diffusion tip may be coupled to or incorporated in one or more therapeutic light output fibers of the therapeutic system. The diffusion tip may be incorporated in a handpiece or other applicator of the therapeutic system.

In some such embodiments, the diffusion tip may be placed in or near a target treatment region of a patient to provide therapeutic light with a desired illumination pattern. The tip may be used externally, incorporated into a surgical instrument for internal use, or introduced into a body cavity of the patient. For example, in one embodiment, the diffusion tip may be introduced in to a periodontal or periimplant pocket of a dental patient to provide illumination in a desired pattern. In another embodiment, the diffusion tip may be introduced into the nares of a patient undergoing treatment to reduce or eliminate a microbial infection in the nasal cavity. In another embodiment, the diffuser tip may be positioned near the finger or toe nails of a patient to apply light used to treat a microbial infection of the nail and/or nail bed.

In various embodiments some or all of the diffuser tip may be constructed of biocompatible and/or autoclavable materials.

In various embodiments, the diffusion tip may be used to apply therapeutic light in a desired illumination pattern for any suitable purpose, including, but not limited to, antimicrobial (e.g., antibacterial, antifungal, antiviral, etc) treatment and thermal treatment (e.g., laser surgical treatments, photothermal or photoablative therapy, thermal coagulation, etc.). Additionally or alternatively, the diffusion tip may be used to apply light to a target region of a patient for other purposes, e.g., medical diagnostic sensing, medical imaging, etc.

The relevant teachings of all references cited herein that enable the claimed inventions are incorporated herein by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. An optical fiber diffusion device comprising:
an optical fiber including a proximal terminus arranged to be coupled to a radiant energy source, and
a distal terminus region including at least one light emission region arranged to emit light from the optical fiber,
the at least one light emission region including at least one crazed diffusion feature formed in the material of the optical fiber itself.

2. An optical fiber diffusion device according to claim 1, wherein the at least one light emission region comprises a plurality of discrete light emission sub-region bands, each light emission sub-region band of the plurality including at least one crazed diffusion feature formed in the material of the optical fiber itself.

3. An optical fiber diffusion device according to claim 1, wherein the at least one light emission region comprises a plurality of discrete optical sub-regions arranged to emit a substantially equal amount of light from each discrete optical sub-region of the plurality.

4. An optical fiber diffusion device according to claim 1, wherein the optical fiber comprises a polymer material.

5. An optical fiber diffusion device according to claim 1, wherein the at least one light emission region comprises optical fiber cladding that is not abraded.

6. An optical fiber diffusion device according to claim 1, wherein the at least one light emission region comprises optical fiber cladding none of which is chemically removed.

7. An optical fiber diffusion device according to claim 1, wherein the at least one light emission region has the same diameter as the diameter of the optical fiber.

8. An optical fiber diffusion device according to claim 1, wherein the at least one light emission region has a smaller diameter than the diameter of the optical fiber.

9. An optical fiber diffusion device according to claim 1, wherein the at least one light emission region comprises at least one elongated emission region.

10. An optical fiber diffusion device according to claim 1, wherein the optical fiber diffusion device comprises no mirror.

11. An optical fiber diffusion device according to claim 1, wherein the optical fiber diffusion device comprises no overtube.

12. An optical fiber diffusion device according to claim 1, wherein the at least one light emission region comprises a plurality of heat-effected light emission sub-regions, each light emission sub-region including necking and crazing of the optical fiber.

13. An optical fiber diffusion device according to claim 1, wherein the at least one light emission region comprises a plurality of light emission sub-regions having logarithmic sub-region spacing.

14. An optical diffusion device according to claim 1, the at least one crazed diffusion feature being the result of heating and elongating the fiber.

15. An optical fiber diffusion device according to claim 1, wherein the at least one crazed diffusion feature is of a configuration that emits light in a fashion that provides substantially uniform illumination of at least one designated object.

16. An optical fiber diffusion device according to claim 15, wherein the at least one light emission region comprises a plurality of discrete light emission sub-region bands, each light emission sub-region band of the plurality including at least one crazed diffusion feature formed in the material of the optical fiber itself, the plurality of discrete light emission sub-region bands being arranged in said configuration that emits light in a fashion that provides substantially uniform illumination of at least one designated object.

17. A method for the manufacture of an optical diffusion device, the method comprising the steps of:
(a) applying a stress to a portion of an optical fiber that includes a location of a light emission region to be formed in the optical fiber;
(b) applying thermal radiation to a sub-region of the portion of the optical fiber that includes the location of the light emission region to be formed in the optical fiber, until a deformation of the sub-region occurs; and
(c) repeating steps (a) and (b) for at least one additional sub-region of the portion of the optical fiber to produce the light emission region in the optical fiber, the light emission region comprising a plurality of discrete light emission sub-region bands formed by the applying of the stress and the applying of the thermal radiation.

18. A method according to claim 17, further comprising, prior to the applying the stress and the applying thermal radiation:
affixing a radiant source to a proximal terminus of the optical fiber;
affixing an optical transmission sensor to a distal terminus of the optical fiber;
clamping the optical fiber at a first proximal position between the radiant source and the location of the light emission region to be formed in the optical fiber; and
clamping the optical fiber at a first distal position between the optical transmission sensor and the location of the emission region to be formed in the optical fiber.

19. A method according to claim 17, further comprising:
controlling at least one of the applying the stress and the applying thermal radiation based on an amount of light transmitted from a distal end of the optical fiber.

20. A method according to claim 19, wherein the controlling is performed based on monitoring the amount of light transmitted from the distal end of the optical fiber to achieve a desired light emission from an effected sub-region of active manufacture, said controlling being based on inversely correlating the amount of light transmitted from the distal end of the optical fiber versus the desired light emission from the effected sub-region of active manufacture.

21. A method according to claim 17 further comprising moving a thermal emitter along the optical fiber to apply the thermal radiation to the at least one additional sub-region.

22. A method according to claim 17, further comprising applying the thermal radiation using a thermal emitter from the group consisting of: a heat gun, a radio frequency device, a light device, a soldering tip, a laser, a coil, and an ultrasound device.

23. A method for the manufacture of an optical diffusion device from a continuous roll of optical fiber, the method comprising:
rolling the optical fiber out of a source roll around which the optical fiber is rolled, such that a region of the optical fiber that is to be formed into at least one light emission region is positioned within a plurality of manufacturing devices to be used in manufacturing the optical diffusion device; and
monitoring light emitted from the fiber during manufacturing using a sensor coupled to the optical fiber.

24. A method according to claim 23, wherein the sensor is coupled to a distal terminus of the optical fiber.

25. A method according to claim 24, wherein the sensor is rotatable, the method further comprising:
receiving the manufactured optical diffusion device using a continuous uptake roll around which manufactured optical fiber is rolled.

26. A method according to claim 23, wherein the monitoring comprises monitoring the amount of light transmitted from the distal end of the optical fiber to achieve a desired light emission from an effected sub-region of active manufacture, said monitoring being based on inversely correlating the amount of light transmitted from the distal end of the optical fiber versus the desired light emission from the effected sub-region of active manufacture.

27. A method according to claim 23, wherein the method comprises manufacturing an optical fiber diffusion device comprising the optical fiber, the optical fiber diffusion device comprising:
the optical fiber, the optical fiber including a proximal terminus arranged to be coupled to a radiant energy source, and
a distal terminus region including the at least one light emission region, the at least one light emission region being arranged to emit light from the optical fiber and comprising a plurality of discrete light emission sub-region bands, each light emission sub-region band of the plurality including at least one crazed diffusion feature formed in the material of the optical fiber itself.

28. An optical fiber diffusion device comprising:
an optical fiber including a proximal terminus arranged to be coupled to a radiant energy source, and a distal terminus region including at least one light emission region arranged to emit light from the optical fiber, the at least one light emission region including at least one crazed diffusion feature formed in the material of the optical fiber itself and
a catheter coupled to the optical fiber, the catheter including a balloon illuminated by light from the optical fiber.

29. An optical fiber diffusion device according to claim 28, wherein the at least one light emission region comprises a plurality of discrete light emission sub-region bands being separated from each other by a distance approximately equal to or less than a radius of the balloon.

30. An optical fiber diffusion device according to claim 28, wherein the at least one crazed diffusion feature is of a configuration that emits light in a fashion that provides substantially uniform illumination of the balloon.

31. An optical fiber diffusion device according to claim 30, wherein the at least one light emission region comprises a plurality of discrete light emission sub-region bands, each light emission sub-region band of the plurality including at least one crazed diffusion feature formed in the material of the optical fiber itself, the plurality of discrete light emission sub-region bands being arranged in said configuration that emits light in a fashion that provides substantially uniform illumination of the balloon.

32. A method of treating the human body, the method comprising:
introducing an optical fiber diffusion device into a vascular vessel of the human body, the optical fiber diffusion device comprising an optical fiber including a proximal terminus arranged to be coupled to a radiant energy source, and a distal terminus region including at least one light emission region arranged to emit light from the optical fiber, the at least one light emission region including at least one crazed diffusion feature formed in the material of the optical fiber itself; and
illuminating the optical fiber diffusion device.

33. A method according to claim 32, wherein the optical fiber diffusion device further comprises a catheter coupled to the optical fiber, the catheter including a balloon illuminated by light from the optical fiber.

34. A method according to claim 33, wherein the method comprises performing a balloon angioplasty.

35. An optical fiber diffusion device comprising:
a source optical fiber including (i) a proximal terminus of the source optical fiber arranged to be coupled to a radiant energy source, and (ii) a distal terminus of the source optical fiber; and
an emission optical fiber including a proximal terminus of the emission optical fiber coupled to the distal terminus of the source optical fiber, the emission optical fiber comprising a distal terminus region including at least one light emission region arranged to emit light from the emission optical fiber, the at least one light emission region including at least one crazed diffusion feature formed in the material of the emission optical fiber itself.

36. An optical fiber diffusion device according to claim 35, wherein the emission optical fiber comprises a disposable tip.

37. An optical fiber diffusion device according to claim 36, wherein the source optical fiber is reusable.

38. An optical fiber diffusion device according to claim 35, wherein the device comprises a handpiece of a dental tool, the handpiece including at least a portion of the source optical fiber.

39. An optical fiber diffusion device according to claim 38, wherein the emission optical fiber is detachably coupled to the source optical fiber.

40. An optical fiber diffusion device according to claim 35, wherein the at least one light emission region comprises a tapered tip.

41. An optical fiber diffusion device according to claim 35, wherein the emission optical fiber comprises a polymer.

42. An optical fiber diffusion device according to claim 35, wherein the source optical fiber comprises a glass fiber.

43. A method of providing treatment light from an optical therapeutic system, the method comprising:
diffusing light from an optical fiber diffusion device in or near a target treatment region of a patient, the optical fiber diffusion device comprising
a source optical fiber including (i) a proximal terminus of the source optical fiber arranged to be coupled to a radiant energy source, and (ii) a distal terminus of the source optical fiber; and
an emission optical fiber including a proximal terminus of the emission optical fiber coupled to the distal terminus of the source optical fiber, the emission optical fiber comprising a distal terminus region including at least one light emission region arranged to emit light from the emission optical fiber, the at least one light emission region including at least one crazed diffusion feature formed in the material of the emission optical fiber itself.

44. A method according to claim 43, the method comprising detachably coupling the emission optical fiber to at least one therapeutic light output fiber of the optical therapeutic system, the at least one therapeutic light output fiber comprising the source optical fiber.

45. A method according to claim 44, wherein the optical fiber diffusion device is incorporated in an applicator of the optical therapeutic system.

46. A method according to claim 43, wherein the emission optical fiber is used external to the body of the patient.

47. A method according to claim 43, wherein the emission optical fiber is incorporated into a surgical instrument for internal use.

48. A method according to claim 43, wherein the emission optical fiber is introduced into a body cavity of the patient.
